SEQUENCE LISTING (10 sequences)

SEQ ID NO: 1 - DNA, 48 bp, Artificial Sequence
  Synthetic
ggggaaaaaaaaggggaaaaaaaaggggaaaaaaaaggggaaaaaaaa

SEQ ID NO: 2 - DNA, 55 bp, Artificial Sequence
  Synthetic
gtcagtcagtcagtcagtcagtcagtcagtcagtcagtcagaaccgaggcgccgc

SEQ ID NO: 3 - DNA, 54 bp, Artificial Sequence
  Synthetic
aaaaaaaaaaaaaaaaaaaacccaaaaaaaaaaaaaaaaaaaaaaaaaaaaaag

SEQ ID NO: 4 - DNA, 53 bp, Artificial Sequence
  Synthetic
gtttttttttttttgttttttttttaaagttttttttttcccgtttttttttt

SEQ ID NO: 5 - DNA, 7 bp, Artificial Sequence
  Synthetic
gattaca

SEQ ID NO: 6 - DNA, 70 bp, Artificial Sequence
  Synthetic
cgccgcggagccaagactgactgactgactgactgactgactgactgactgactgttgcatgtcctgtga

SEQ ID NO: 7 - DNA, 15 bp, Artificial Sequence
  Synthetic
tcacaggacatgcaa

SEQ ID NO: 8 - DNA, 55 bp, Artificial Sequence
  Synthetic
  misc_feature (1)..(55): n = 7-deaza dGTP
cantcantcantcantcantcantcantcantcantcantcttnnctccncnncn

SEQ ID NO: 9 - DNA, 54 bp, Artificial Sequence
  Synthetic
  misc_feature (1)..(54): n = 7-deaza dGTP
ttttttttttttttttttttnnnttttttttttttttttttttttttttttttc

SEQ ID NO: 10 - DNA, 53 bp, Artificial Sequence
  Synthetic
  misc_feature (1)..(53): n = 7-deaza dGTP
ttttttttttttttttttttnnntttttttttttttttttttttttttttttt

(19) United States
(12) Patent Application Publication
(10) Pub. No.: US 2021/0139959 A1
(43) Pub. Date: May 13, 2021

(54) MODIFIED NUCLEOTIDE TRIPHOSPHATES FOR MOLECULAR ELECTRONIC SENSORS

(71) Applicant: Roswell Biotechnologies, Inc., San Diego, CA (US)

(72) Inventors: Barry L. Merriman, San Diego, CA (US); Tim Geiser, San Diego, CA (US); Paul W. Mola, San Diego, CA (US); Darryl Rideout, San Diego, CA (US)

(21) Appl. No.: 16/321,784

(22) PCT Filed: Aug. 1, 2017
(86) PCT No.: PCT/US2017/044965

§ 371 Date: Jan. 29, 2019

Related U.S. Application Data
(60) Provisional application No. 62/452,466, filed on Jan. 31, 2017.
(60) Provisional application No. 62/369,696, filed on Aug. 1, 2016.

(51) Int. Cl. C12Q 1/6825; C07H 19/207; C07H 19/10; C12Q 1/682; C12Q 1/6874; G01N 27/414

(52) U.S. Cl. CPC C12Q 1/6825; C07H 19/207; G01N 27/4145; C12Q 1/682; C12Q 1/6874; C07H 19/10

(57) ABSTRACT

In various embodiments, a signal enhancement process for single molecule molecular sensors is disclosed along with new modified dNTPs. In general, the enhancement process comprises providing a molecular electronic sensor comprising a polymerase and providing a nucleotide template and modified dNTPs in appropriate buffers to the polymerase, wherein incorporation of the modified dNTPs during action of the polymerase enzyme provides an enhanced electrical signal or improved signal-to-noise ratio for the incorporation event relative to the performance with corresponding native dNTPs.

### CROSS-REFERENCE TO RELATED APPLICATIONS

This application claims priority to U.S. Provisional Pat. App. Ser. No. 62/369,696, filed Aug. 1, 2016, entitled “Modified Nucleotides for Molecular Sensors,” and U.S. Provisional Pat. App. Ser. No. 62/452,466, filed Jan. 31, 2017, entitled “Modified Nucleotide Triphosphates for Molecular Electronic Sensors,” the disclosures of which are incorporated herein by reference in their entireties.

### FIELD

The present disclosure relates generally to the field of molecular electronic biosensors, and particularly to the design and use of modified deoxynucleotide triphosphates (dNTPs) to enhance signals produced by molecular electronic sensors.

### BACKGROUND

Molecular electronics generally refers to the use of single molecules or molecular assemblies as components of an electronic circuit. In particular, such a circuit may comprise a sensing circuit in which the single molecules constitute the transducer that interacts with a test solution to produce an electrical signal related to the composition of the test solution. Of concern are applications where a sensor complex comprises a polymerase enzyme to provide the capability to sense properties of DNA and/or RNA molecules in a test solution. In this type of bio-sensor, the polymerase interacts with a DNA or RNA molecule as a template for polymerization of a complementary strand, which the polymerase produces by incorporating dNTPs provided in the test solution, and in so doing modulates the parameters of a molecular circuit to produce electrical signals. For this class of polymerase-based biosensors, one important factor of the performance is dNTP content of the solution.

In spite of the advancements in molecular sensors and the level of sophistication of molecular sensors comprising a polymerase enzyme, the overall signal, and/or signal-to-noise-ratio (SNR) related to nucleotide incorporation events may not be sufficient to distinguish between dNTPs or to detect particular molecular events. Hence, further improvements to these sensors is continuously warranted, including possible modifications to dNTPs that may improve overall signal and/or signal-to-noise-ratio in molecular sensors comprising polymerase enzyme.

### SUMMARY OF THE INVENTION

In various embodiments of the present disclosure, modified dNTPs, synthesis of modified dNTPs, and use of modified dNTPs in molecular sensors are disclosed. Modified dNTPs in accordance to the present disclosure are shown to improve signaling performance in molecular sensors, including enhancing the overall signal, providing distinguishing signal shape, and/or improving signal-to-noise-ratio related to nucleotide incorporation events in molecular sensors comprising a polymerase enzyme.

In various embodiments, a modified nucleotide (modified dNTP) is disclosed. The modified nucleotide comprises a structure represented by compound [16],

[chemical structure]

wherein: Nuc is A, T, C, or G; Y is selected from O, S, B or I; n is an integer from 2 to 5; and
R¹ is selected from:

[chemical structure]

[chemical structure]

[chemical structure]

[chemical structure]

[chemical structure]

wherein n=1 to 100, and wherein the modified nucleotide is incorporated by DNA polymerase in replication of a DNA template during DNA sequencing. In various embodiments, the modified dNTP may comprise a tetra-phosphate or a hexa-phosphate moiety instead of the natural triphosphate moiety.

In various aspects, the modified nucleotide is compound [16] wherein Y is O, Nuc is cytosine, n is 3, and R¹ is the substituent:

[chemical structure]

In other aspects, the modified nucleotide is compound [16] wherein Y is O, Nuc is cytosine, n is 3, and R¹ is the substituent:

[chemical structure]

In certain aspects, the modified nucleotide is compound [16] wherein Y is O, Nuc is cytosine, n is 3, and R¹ is the substituent:

[chemical structure]

In other embodiments, the modified nucleotide is compound [16] wherein Y is O, Nuc is cytosine, n is 3, and R¹ is the substituent:

[chemical structure]

In various examples, the modified nucleotide is compound [16] wherein Y is O, Nuc is cytosine. n is 3, and R¹ is the substituent:

[chemical structure]

In other aspects, the modified nucleotide is compound [16] wherein Y is O, Nuc is cytosine, n is 5, and R¹ is the substituent:

[chemical structure]

In various embodiments, the modified nucleotide is compound [16] wherein Y is O, Nuc is cytosine, n is 5, and R¹ is the substituent:

[chemical structure]

In some examples, the modified nucleotide is compound [16] wherein Y is O, Nuc is cytosine, n is 5, and R¹ is the substituent:

[chemical structure]

In various aspects, the modified nucleotide is compound [16] wherein Y is O, Nuc is cytosine, n is 5, and R¹ is the substituent:

[chemical structure]

In some examples, the modified nucleotide is compound [16] wherein Y is O, Nuc is cytosine, n is 5, and R¹ is the substituent:

[chemical structure]

In various embodiments, the modified nucleotide is compound [16] wherein Y is O, Nuc is cytosine, n is 5, and R¹ is the substituent:

[chemical structure]

In other aspects, the modified nucleotide is compound [16] wherein Y is O, Nuc is adenosine, n is 3, and R¹ is the substituent:

[chemical structure]

In certain embodiments, the modified nucleotide is compound [16] wherein Y is O, Nuc is cytosine, n is 3, and R¹ is the substituent:

[chemical structure]

In various embodiments, the modified nucleotide is compound [16] wherein Y is O, Nuc is adenosine, n is 3, and R¹ is the substituent:

[chemical structure]

wherein n=1 to 100.

In certain aspects, the modified nucleotide is compound [16] wherein Y is O, Nuc is adenosine, n is 3, and R¹ is the substituent:

[chemical structure]

In various embodiments of the present disclosure, a modified nucleotide (modified dNTP) is described. The modified nucleotide structure is represented by compound [18],

[chemical structure]

wherein:
Nuc is a DNA base selected from A, T, C, and G;
Y is selected from OH, SH, or BH₃;
n is an integer from 2 to 5;
R³ is selected from H or halogen;
R¹ is selected from H, linear or branched C₁-C₅ alkyl, C₃-C₈ cycloalkyl, or aryl, optionally substituted with halogen, Me or OMe, or —(CH₂CH₂O)ₓMe wherein x is an integer 1 to 20;
L² is selected from —(CH₂)_{q}— wherein q is an integer from 1 to 10, —CH₂CH₂(OCH₂CH₂)_{y}— (wherein y is an integer from 1 to about 8), —(CH₂)_{q}—O—(CH₂CH₂O)_{y}—CH₂— (wherein q is an integer from 1 to about 10 and y is an integer from 1 to about 8), —CO(CH₂)ᵣ— wherein r is an integer from 1 to about 10), —COCH₂CH₂(OCH₂CH₂)_{z}— wherein z is an integer from 1 to 6, —COCH₂CH₂CONH(CH₂)ₘ— wherein m is an integer from 1 to 6, —COCH₂CH₂CONH(CH₂CH₂O)ₚCH₂CH₂— wherein p is an integer from 1 to 6, 1,4-benzenediyl, 1,3-benzenediyl, or 1,2-benzenediyl, with carbon atoms optionally and independently substituted with halogen, Me, Et, OH, OMe, or CF₃, or is,

[chemical structure]

and
R⁴ and R⁵ are independently selected from H, phenyl,

[chemical structure]

wherein n=1 to 100.

In various aspects, the modified nucleotide is compound [18] wherein: Nuc is A, T, G, or C; Y is OH; n=3 or 5; R¹ is H; R³ is H; L² is the bivalent linker —(CH₂)₄—O—(CH₂CH₂O)₈—CH₂—, and R⁴ is selected from phenyl,

[chemical structure]

In various embodiments of the present disclosure, a method of enhancing an electrical signal generated from a biosensor is described. The method comprises: (a) providing a biosensor comprising source and drain electrodes and a polymerase bonded to a bridge molecule bridging the electrodes to complete an electrical circuit; (b) placing a nucleotide template to be sequenced in communication with the polymerase; (c) placing a modified dNTP in communication with the polymerase; and (d) transcribing the nucleotide template by the polymerase, wherein transcribing comprises incorporating the modified dNTP by the polymerase, and wherein incorporating the modified dNTP results in an enhanced electrical signal compared to incorporating the corresponding non-modified dNTP. In some instances, the enhanced signaling comprises at least one of a larger current spike, a distinguishing current v. time peak shape, or improved signal-to-noise ratio.

In certain examples, the modified dNTP may comprise any one of the modified nucleotides disclosed herein. Further, the enhanced electrical signal made possible by the modified dNTP distinguishes between A, G, C, and T in the nucleotide template, making sequencing of the nucleotide template more reliable. In other examples, the enhanced electrical signal is unique for incorporation of each of a modified dATP, a modified dGTP, a modified dTTP, and a modified dCTP.

In various embodiments of the present disclosure, a method of transcribing a nucleotide template is described. The method comprises: (a) providing a polymerase capable of transcribing the nucleotide template; (b) placing the nucleotide template in communication with the polymerase; (c) placing modified dNTPs in communication with the polymerase; and (d) transcribing the nucleotide template with the polymerase by incorporating the modified dNTPs, wherein the modified dNTPs comprise any one of the modified nucleotides disclosed herein.

### BRIEF DESCRIPTION OF THE DRAWING FIGURES

FIG. 1 is a schematic illustration of an embodiment of a molecular electronic bio-sensor for DNA sequencing;

FIG. 2 illustrates an embodiment of a molecular sensor structure 200 comprising a polymerase;

FIG. 3 illustrates a schematic of an embodiment of a test set-up for electrical measurements on molecular sensors;

FIG. 4 depicts electron microscope images of metal electrodes pairs further comprising gold metal dot contacts usable for binding bridge molecules.

FIGS. 5A and 5B illustrate use of modified dNTPs to enhance the signaling from a polymerase-based sensor. FIG. 5A illustrates a sensor in operation with standard dNTPs (at left) and with modified dNTPs (at right). FIG. 5B illustrates an exemplary current versus time plot recorded from the sensor in operation with standard dNTPs (at left) and in operation with modified dNTPs (at right);

FIGS. 6A and 6B illustrate sequencing signals obtained from a polymerase sensor while processing the template with sequence G₄A₈-G₄A₈-G₄A₈-G₄A₈ (SEQ ID NO: 1) comprising standard dNTPs;

FIG. 7 illustrates sequencing signals with modified dNTPs. The signals were obtained for the DNA template {GTCA}₁₀-GAACCGAGGCGCCGC (SEQ ID NO: 2), comprising a modified dGTP (namely, deaza dGTP);

FIGS. 8A and 8B illustrate another embodiment of sequencing signals with modified dNTPs. The sequencing signal data was obtained for the DNA template {GTCA}₁₀-GAACCGAGGCGCCGC (SEQ ID NO: 2), comprising a modified dGTP (deaza dGTP);

FIG. 9 depicts another embodiment of sequencing signals with modified dNTPs. The sequencing signal data was obtained for the DNA template A₂₀-C₃-A₃₀-G (SEQ ID NO: 3), comprising a modified dGTP (deaza dGTP);

FIG. 10 depicts another embodiment of sequencing signals with modified dNTPs. The sequencing signal data was obtained for the DNA template (GT₁₀-T₃-GT₁₀-A₃-GT₁₀-C₃-GT₁₀) (SEQ ID NO: 4), comprising a gamma-phosphate modified dCTP (dC4P-Lactose and dC4P-Cy7 mixed in equal amounts);

FIG. 11 sets forth mechanisms by which modified dNTPs can enhance signals from polymerase activity in a molecular electronic sensor;

FIG. 12 sets forth the standard dNTPs and indicates (by “star” locations) on the chemical structures where modifications can be made that are well tolerated by polymerase enzymes;

FIGS. 13A, 13B, 13C and 13D illustrate embodiments of modified dNTPs, with “stars” indicating the site(s) of modification from the natural dNTP;

FIGS. 14A and 14B illustrate embodiments of modified dNTPs comprising deaza purine structures, with the “star” at the 7-position indicating the location where a C atom replaced a N atom;

FIGS. 15A, 15B and 15C illustrate embodiments of modified dNTPs comprising various derivatizations of the γ-phosphate of the triphosphate, illustrating the ability to add increments of Coulombic charge to a natural dNTP;

FIG. 16 illustrates an embodiment of a general class of modified dNTP compounds;

FIG. 17 illustrates another embodiment of a general class of modified dNTP compounds;

FIG. 18 illustrates another embodiment of a general class of modified dNTP structures;

FIG. 19 illustrates options for the bivalent linker moiety L¹ found on the genus structure [17] in FIG. 17;

FIGS. 20A-20F illustrate specific embodiments of γ-modified dCTPs;

FIG. 21 is a gel image from a polymerase extension assay used as a functional test of modified dNTPs;

FIG. 22 illustrates a synthesis of DBCO-PEG (compound [IV;

FIG. 23 illustrates a synthesis of DBCO-PEG-monophosphate (compound [V]);

FIGS. 24A-24B illustrate a synthesis of dC4P-DBCO (compound [VIII];

FIGS. 25A-25B illustrate a synthesis of dCTP-pipDMA (compound [X]);

FIGS. 26A-26B illustrate a synthesis of dCTP-Cy7 (compound [XII]);

FIGS. 27A-27B illustrate a synthesis of dCTP-TPMD (compound [XIV]);

FIGS. 28A-28B illustrate a synthesis of dCTP-Lactose (compound [XVI]);

FIGS. 29A-29B illustrate a synthesis of dCTP-PEG9 (compound [XVIII]);

FIGS. 30A-30B illustrate two embodiments of R¹ substituents that provide direct interaction of positive charges on R¹ with the conducting portion of a molecular sensor;

FIGS. 30C-30D illustrate two embodiments of R¹ substituents that provide direct interaction of negative charges on R¹ with the conducting portion of a molecular sensor;

FIGS. 31A-31D illustrate four embodiments of R¹ substituents that provide direct 7E-7E and hydrophobic interaction of aromatic rings on R¹ with the conducting portion of a molecular sensor;

FIGS. 32A-32B illustrate two embodiments of R¹ substituents that provide both charge and π-interaction between a modified dNTP carrying the illustrated R¹ substituent and the conducting portion of a molecular sensor;

FIGS. 33A-33B illustrate two embodiments of R¹ substituents that provide radical anion charge or radical cation charge interaction between a modified dNTP carrying the illustrated R¹ substituent and the conducting portion of a molecular sensor;

FIG. 34 illustrates an embodiment of an R¹ substituent that provides a mechanism for unique electrical signal generation through creating and breaking a conductive link between two conductors;

FIG. 35A illustrates an embodiment of a modified dATP;

FIG. 35B illustrates an embodiment of a special tether than can be covalently bonded between a polymerase and a bridge molecule in a molecular sensor, where it can be available to interact with various modified dNTPs during incorporation events;

FIG. 36 depicts a modified dATP capable of altering the conformational change of a polymerase during incorporation of same; and

FIG. 37 illustrates the chemical structures of various modified dNTPs along with a gel image from a polymerase extension assay used to test these modified dNTPs.

### DETAILED DESCRIPTION

In various embodiments of the present disclosure, signaling performance of a polymerase-based molecular electronic sensor is enhanced by the use of modified dNTPs carried in a test solution analyzed by the sensor. Many such dNTP modifications are tolerated by a polymerase in a sensor. In certain aspects, signal enhancements provided by modified dNTPs can occur through a variety of mechanisms, as discussed herein, and these mechanisms can be used as a template for rational design of such modifications.

In various embodiments, a molecular sensor usable for DNA sequencing comprises a polymerase enzyme to functionalize the sensor. The sensor also comprises a conducting bridge molecule (also referred to as a “molecular wire”). In some aspects, a conducting bridge molecule may be on the order of about 10 nm in length. The conducting bridge molecule “wires” the conjugated polymerase into a circuit comprising source and drain electrodes and the molecular wire (a molecule) bridging the electrode pair. Such a molecular wire may comprise a DNA oligonucleotide (“oligo”), protein alpha helix bridge, or other biomolecule that connects source and drain electrodes. In certain aspects, a polymerase enzyme coupled to a molecular wire element completes a current measuring circuit in the sensor. Measurement of current versus time as the polymerase incorporates nucleotides produces a signal trace that indicates the incorporation events as discrete signal spikes, (e.g., current spikes in a trace of current versus time), and discriminates and identifies the different bases being incorporated via the detailed shape and/or size of these spikes. The resulting signal is processed to determine the sequence of the template.

The critical enzyme activity monitored by such a molecular sensor is the incorporation of the various deoxynucleotide tri-phosphates (or “dNTPs”). A subject of this disclosure is, in general, an alteration of this process in order to enhance the resulting signals, which consequently improves the ability of a molecular sensor based on polymerase to determine a DNA sequence.

dNTPs

The four specific forms of dNTPs correspond to the four bases/letters of DNA, which are dATP (adenosine), dCTP (cytidine), dGTP (guanosine) and dTTP (thymidine), which differ only in the base composition. Herein, the base on a molecule may be denoted by a letter (A, C, G, T) or in general, by “Nu” or “Nuc.” All of the natural dNTPs have a triphosphate moiety, and the three phosphate groups are indicated as α, β, and γ, beginning with the phosphate attached to the 5′ OH of the deoxyribose. In a DNA chain extension that is catalyzed by polymerase, the 3′ OH group on the deoxyribose and the 5′ α-phosphate site participate in an incorporation and chain extension. The four specific dNTPs are the chemical substrates that DNA polymerase incorporates into a growing strand, guided by a template strand. The most critical feature of this process is that the β- and γ-phosphate groups are released together as a pyrophosphate group by the action of the polymerase enzyme, when the 3′ OH group of the elongating strand is coupled to the 5′ carbon-α-phosphate group of the incoming nucleotide.

The extent to which natural dNTPs can be chemically modified yet still be recognized and incorporated by a polymerase enzyme during polymerase chain reaction (PCR) remains largely unknown. In general, modifications to the 5′ carbon/α-phosphate or 3′ OH group are very limited, since these sites engage in critical coupling reactions in forming the chain. All other sites on the dNTP typically allow some degree of chemical modification. This may or may not result in native form DNA being produced, depending on whether the modifications reside on the β- and/or γ-phosphate groups released from the dNTP by the polymerase during incorporation, or elsewhere on the base, deoxyribose group or the α-phosphate that are retained in the growing strand. Specific examples include a tolerated dNTP having a large dye-label group attached to the base (see Waggoner, et al, Nucl. Acids Res. (1994) 22 (16): 3418-3422.). This modification is retained in the resulting DNA product. A specific example of modification to the γ-phosphate, in which linker and dye molecules can be attached without inhibiting the polymerase, is found in Fuller, et al, Nucleosides, Nucleotides, and Nucleic Acids, 24 (5-7):401-408, (2005). These modifications are not retained in the resulting DNA product. In general, the incorporation of dye-labeled dNTPs has been the basis of many methods of analyzing DNA using optical reporter strategies.

Disclosed herein are broad classes of dNTP modifications. These modifications are generally to the β- and/or γ-phosphate such that the modifications are not retained in the DNA synthesized by the polymerase from the modified dNTPs, although some modifications herein are also to the α-phosphate group and are retained in the synthesized DNA. In various embodiments, these forms may comprise a linker such as from the γ-phosphate to a signaling group. The signaling group may comprise a group that is charged (e.g., +1, +2, or −1, −2, etc.) under the buffering conditions used for sensor operation, such that its charge impacts current flow in the sensor circuit. For example, a signaling group may comprise a sulfonate group (—SO₃⁻, with −1 charge), or a quaternary ammonium substituent (—R₃N⁺ with a +1 charge). Other signaling groups attached to dNTPs may comprise dyes, sugars, polycyclic aromatic substituents, or other groups. In certain aspects, a linker enables the signaling group to come into close proximity to the molecular bridge of the sensor during incorporation of the modified dNTP carrying the linker and signaling group, thereby enhancing signal impact. There may be different signaling groups for the different bases, such as to enhance base discrimination of signals. The chemical structures of modified dNTPs, their synthesis via synthetic organic chemistry methods, and their use in molecular sensors based on polymerase are detailed herein.

It has been determined that modified dNTPs are especially effective when the molecular sensor is on the order of 10 nm in size, as illustrated in FIG. 2, so as to reduce the noise and allow charge alterations provided by the various dNTPs to influence a larger portion of the molecular circuit. For purposes herein, the size of the molecular sensor can be in the range of from about 3 nm to about 30 nm, in the range of from about 5 nm to about 15 nm, or in the range of from about 6 nm to about 12 nm.

Exemplary Molecular Electronic Biosensors for DNA Sequencing

As used herein, various chemical compounds may be labeled and identified by an Arabic number, (e.g., compounds [1], [2], [3], etc.), Other compounds may be labeled and identified by a Roman numeral, (e.g., compounds [VI], [VII], [VIII], etc.). A compound labeled with an Arabic number is distinct from a compound labeled with the equivalent Roman numeral. As an example, compound [16] and compound [XVI] are different compounds.

As used herein, sequences with repeating bases may be written in shorthand notation for convenience, wherein a subscripted integer indicates the total number of a particular base immediately preceding the subscripted integer. For example, the shorthand notation A₂₀-C₃-A₃₀-G (SEQ ID NO: 3) denotes the sequence, A-A-A-A-A-A-A-A-A-A-A-A-A-A-A-A-A-A-A-A-C-C-C-A-A-A-A-A-A-A-A-A-A-A-A-A-A-A-A-A-A-A-A-A-A-A-A-A-A-A-A-A-A-G (SEQ ID NO: 3).

FIG. 1 illustrates an embodiment of a molecular electronic bio-sensor for DNA sequencing. Here, a molecular complex comprises an enzyme such as polymerase that engages with a DNA strand to form a molecular electronic circuit with a sensitive current meter. The polymerase of the sensor engages with a DNA template to transcribe it, resulting in electrical current signals over time as the individual dNTPs are incorporated. Exemplary signaling is shown in FIG. 1 in the current versus time plot directly under the schematic of the sensor. The measured current signals ideally contain distinctive spikes that correspond to the different DNA bases, allowing the base sequence to be determined. The example depicted in FIG. 1 shows that the base sequence GATTACA (SEQ ID NO: 5) was deduced from the current spikes in the plot of current versus time.

FIG. 2 illustrates an embodiment of a molecular sensor structure 200 comprising a polymerase, which can be used for testing various modified dNTPs. Molecular sensor structure 200 comprises two electrodes 201 and 202. Electrodes 201 and 202 may comprise source and drain electrodes in a circuit. The electrodes 201 and 202 are separated by a nanogap of about 10 nm. Other gap distances may be required to accommodate other lengths of biomolecular bridges. In this example, the bridge molecule 203 comprises a double-stranded DNA molecule of about 20 nm in length (e.g., 60 bases), with thiol groups 204 and 205 at both the 3′ and 5′ ends for coupling of the bridge molecule 203 to gold contacts 206 and 207 provided on each metal electrode 201 and 202. The probe molecule in this case comprises polymerase 210, e.g., E. coli Pol I, chemically crosslinked at covalent linkage 211 to a Streptavidin protein 212, which in turn is coupled to a binding site 214 via a biotinylated nucleotide in the synthetic DNA oligo 203. In operation, the sensor 200 further comprises a DNA strand 220 being processed by the polymerase 210. The figure approximates the relative sizes of the molecules and atoms.

FIG. 3 illustrates an embodiment of various electrical components and connections in molecular sensors. In the upper portion of the figure, a cross-section of an electrode-substrate structure 300 is illustrated, with attachment to an analyzer 301 for applying voltages and measuring currents through the bridge molecule of the sensor. In the lower portion of the figure, a perspective view of electrode array 302 is illustrated, usable for bridging circuits. Each pair of electrodes comprises a first metal (e.g., “Metal-1”), and a contact dot or island of a second metal (e.g., “Metal-2”) at each electrode end near the gap separating the electrodes. In other examples, Metal-1 and Metal-2 may comprise the same metal. In other aspects, the contact dots are gold (Au) islands atop metal electrodes comprising a different metal. In various experiments, contact dots comprise gold (Au) beads or gold (Au)-coated electrode tips that support self-assembly of a single bridge molecule over each gap between electrode pairs, such as via thiol-gold binding.

FIG. 4 depicts electron microscope images (A), (B), and (C) of metal electrodes with gold metal dot contacts usable for bridge binding. Electrodes are on a silicon substrate, e.g., produced via e-beam lithography. The image at (A) shows an array of titanium electrodes with gold dot contacts. As shown, the width of each electrode in a pair of electrodes is about 20 nm. The image at (B) is a close-up of an electrode gap of about 7 nm, with the gold dot contacts spaced apart by about 15 nm. The image at (C) is a further close-up of a single electrode pair and the features of the nanogap. The image clearly shows the nanogap between electrodes to be about 7 nm and the gold dot spacing to be about 10 nm. As seen in the EM image, the gold dots are positioned adjacent to the nanogap.

Use of Modified dNTPs for DNA Sequencing Applications

FIG. 5A illustrates a schematic of a sensor in operation with a variety of standard dNTPs in solution around the polymerase of the sensor. The illustration at the right of FIG. 5A shows the same sensor but with modified dNTPs in solution around the polymerase. At left in FIG. 5A, an embodiment of a molecular electronic DNA sequence sensor comprises a polymerase mounted on a bridge molecule to complete an electrical circuit, wherein the processing of a template by incorporating dNTPs in solution produces spikes that indicate incorporation events and the identity of the incorporated base.

FIG. 5B illustrates improved signaling possible by using modified dNTPs instead of standard dNTPs. At the left of FIG. 5B is a plot of current versus time recorded during molecular incorporation events involving only native dNTPs. Such current spikes as shown may be difficult to discern from noise, and from each other. At the right of FIG. 5B is a plot of current versus time during molecular incorporation events that include modified dNTPs. The plot at the right of FIG. 5B illustrates that modified nucleotides can enhance signaling to provide clearer signals for distinct incorporation events, and provide signals that discriminate the different bases being incorporated, thereby sensing the sequence. In various aspects, enhanced signaling may comprise larger current spikes and/or different current spike shapes.

In some examples, modifications to dNTPs comprise the addition of various groups to the γ-phosphate of the molecule. The modifications may comprise chemical moieties that provide a formal charge (e.g., +1, −1, etc.), polarity (e.g., —C═O, —OH functionality), non-polar character (e.g., hydrophobicity through use of non-polar functionality like —CH₂—), steric effects (e.g., large fused ring systems). These, and other, chemical moieties can interact with native features on the polymerase enzyme, with engineered features on the polymerase, with the bridge molecule, or with a molecule bonded between the polymerase and bridge molecule. In various aspects, modifications to dNTPs may extend the polyphosphate charge from −3 (in native dNTPs) to −4, −5, −6, −7, −8 or −9, for example, depending on the total number of phosphate groups. In certain embodiments, an initial modification to a dNTP may comprise addition of a click chemistry group to the end of the polyphosphate chain, which provides a reactive moiety (e.g., an alkyne group) usable to efficiently synthesize the target modified dNTP.

In various embodiments, modified dNTPs may require an appropriate buffer solution in which to be effective for signal enhancement, relative to standard buffering conditions commonly used for polymerase reactions (such as PCR, primer extension, or reverse transcription reactions, or the in vivo conditions in various biological organisms wherein these enzymes function). In particular, buffer modifications for some embodiments may include an alteration of the salt concentration used in the buffer, to either higher salt or lower salt conditions. In certain embodiments, reduction of the salt concentration by the range of 2-fold, to 10-fold, to 100-fold, to 1000-fold, 1 million fold, up to 1 billion fold may be advantageous. Advantages may be due to decreasing the salt-based screening of electrical fields in the solution, or the increase in Debye length in the solution, or also due to the decrease in electrical measurement noise from ionic conduction from such lower ion concentrations. In other examples, increase in the salt concentration by the range of 2-fold, to 10-fold, to 100-fold, to 1000-fold, 1 million fold, up to 1 billion fold may be advantageous.

In certain aspects, alterations to the sensor system in general may enhance the effect of modified dNTPs. These alterations can include, but are not limited to, optimization of the concentration of dNTPs, the nature of the buffer solution used, the applied electrode and gate voltages, and the specific type of polymerase or mutated polymerase used in the sensor. Any combination of these alterations may be used to enhance the effects made possible by modified dNTPs. For example, lowering the ion concentration of the buffer in order to extend the Debye radius of charged groups on a modified dNTP may have enhancing effect by allowing greater electrical influence of the dNTP on the molecular bridge conductor during incorporation events.

In other aspects, a buffer may have an altered concentration or composition of metal multivalent cations required for enzyme activity. For example, divalent cations are known to play critical roles in mediating the interaction between the dNTP and a polymerase. Buffer alterations related to this may include alteration of the Mg concentration, or may comprise use of concentrations of other metal multivalent cations, for example divalent cations such as Mn, Fe, Ni, Zn, Co, Ca, Cd, Ba, Sr, Cu, or Cr. The addition of detergents or dispersing agents in the buffer may be important for some embodiments, to reduce or prevent aggregation of modified dNTPs, or reduce or prevent their aggregation with other molecules in the system.

FIGS. 6A and 6B illustrate an embodiment of experimental sequencing signals obtained from natural dNTP incorporation events in a biosensor comprising a polymerase. These signals from the polymerase sensor are obtained by processing the template with sequence G₄A₈-G₄A₈-G₄A₈-G₄A₈ (SEQ ID NO: 1). The signals show electrical current versus time during an experimental run. FIG. 6A also shows a dashed circle indicating an expanded inset found in FIG. 6B. The inset illustrated in FIG. 6B is the portion of the plot from about t=34 seconds to about t=39 seconds, which shows electrical signals reflective of the underlying sequence. Enhancing such signals would provide for more accurate sequencing.

FIG. 7 shows the result of using modified dNTPs. In this example, 7-deaza-dGTP (see FIG. 14B) was used as a modified dGTP. The signals were obtained for the DNA template {GTCA}₁₀-GAACCGAGGCGCCGC (SEQ ID NO: 2), comprising the 7-deaza modified dGTP. Incorporations of this modified dGTP against the C bases of the template may result in enhanced signals.

FIG. 8 depicts another embodiment of sequencing sensing when modified dNTPs are used. In this embodiment, the signal data are obtained for the DNA template {GTCA}₁₀-GAACCGAGGCGCCGC (SEQ ID NO: 2), when a modified dGTP is used (7-deaza dGTP of FIG. 14B). Incorporations of this against the C bases of the template may result in enhanced signal, as shown in the inset of FIG. 8B wherein the portion of the plot from about t=33 seconds to about t=37.5 seconds is expanded. FIG. 8B shows an interpretation of the data with possible enhancement of GG incorporation signals that may result from the use of the modified dGTP.

FIG. 9 depicts another embodiment of sequencing sensing with modified dNTPs. This plot shows sequencing signal data for the DNA template A₂₀-C₃-A₃₀-G (SEQ ID NO: 3), when a modified dGTP is used (7-deaza dGTP of FIG. 14B). Incorporations of this against the C bases of the template may result in enhanced signaling. FIG. 9 shows an interpretation of the data where the signal from T incorporations is discriminated from that of the modified dGTP incorporations, wherein the T signals are increased current, and the modified dGTP signals are seen as a reduction in current. If these signals become convolved, it can produce the net signal seen, with a dip in current in the middle of a broader enhanced current, as the effect of the modified dGTP (see expanded plot within square inset for these details).

FIG. 10 depicts another embodiment of sequencing sensing with modified dNTPs. This plot shows sequencing signal data for the DNA template (GT₁₀-T₃-GT₁₀-A₃-GT₁₀-C₃-GT₁₀) (SEQ ID NO: 4), when γ-phosphate modified dCTPs are used, namely an equal mixture of dC4P-Lactose (compound [XVI] in FIG. 20F and FIG. 28B) and dC4P-Cy7 (compound [XII] in FIG. 20B and FIG. 26B). Incorporations of these modified dCTPs against the G bases of the template may result in an enhanced signal. The plot in FIG. 10 shows current versus time data over multiple incorporation events, seen as current spikes in the data near t=20 seconds. These signal spike heights are potentially enhanced by the use of the modified dCTP forms.

With reference now to FIG. 11, possible mechanisms by which modified dNTPs can enhance signals obtained during polymerase activity in a molecular electronic sensor are illustrated. Possible mechanisms include, but are not limited to: (A) adding local charge (+ or −) to gate the current; (B) slowing down of the rate of polymerase action while a template is bound; (C) changing the conformation of the polymerase, e.g., increasing the finger motion in a portion of the enzyme as shown; and (D) making direct interactions with the bridge molecule to modulate the current. In mechanism (D), the direct interaction of dNTP and bridge molecule can be made possible by tethering an appendage of suitable length from a dNTP to make a modified dNTP that can trail the tethered appendage into direct contact with the bridge molecule while interacting with the polymerase enzyme.

In various embodiments, the use of modified dNTPs in DNA sequencing with a biosensor is disclosed. In DNA or genome sequencing applications, for example, a bridge molecule, such as a DNA oligonucleotide, is conjugated to a polymerase, the polymerase is bound with a primed single-stranded template DNA, and the electronic biosensor is provided with a buffer containing dNTPs (deoxynucleotide triphosphates) for incorporation. Electric current through the bridge molecule is monitored as the various dNTPs are incorporated by the polymerase in synthesizing the complementary strand of the template DNA. In various embodiments, native or “standard” dNTPs are used, including dATP, dCTP, dGTP, and dTTP. However, in other aspects, any or all of these standard dNTPs may be replaced by a corresponding modified dNTP.

FIG. 12 illustrates the standard (“natural”) dNTPs along with locations where various molecular modifications can be made to the dNTP that may enhance the incorporation signals of the dNTP, or that may produce signals with enhanced differences between the different base (A, C, G, T) incorporation events. Non-limiting sites for possible chemical modification of the dNTPs are indicated by a “star.” The stars indicate the sites where modification to the dNTP can be made without affecting the ability for the dNTP to be incorporated and extended by a polymerase. In various embodiments, modified dNTPs comprise one or more modifications at these sites. At left in the figure, allowed sites on the polyphosphate chain (e.g., α, β, γ) or sugar (e.g., the circled 3′ OH group available for derivatization) are depicted. Three stars at the left of a dNTP indicate that modifications can be made at any one of the three phosphate groups, in any combination. At the right of FIG. 12, the two bases purine and pyrimidine are drawn with stars indicating the location of generally allowed sites for modification. For example, the 6- and/or 7-positions on the purine may be derivatized, or the 2- and/or 5-positions of the pyrimidine may be derivatized, amongst other locations on each dNTP. In some examples, a 7-deaza modification is utilized. In various examples, greater accuracy in determining the template sequence is provided, (e.g., as illustrated in FIGS. 5A and 5B).

Non-limiting examples of dNTP modifications in accordance to the present disclosure are shown in FIGS. 13 through 20. The dNTP modifications include, but are not limited to, modifications of the base, such as to produce 7-deaza forms or 8-bromo forms, modification to the α- or β-phosphate groups, such as to produce thiolated forms or brominated derivatives of these phosphates, or γ-phosphate modifications, including the addition of extra phosphate groups, such as to form tetra-, penta- or hexa-phosphates, or even longer phosphate chains from the original triphosphate group. Polymerase is highly tolerant of many diverse groups added to the γ-phosphate. Modification of dNTPs by adding another group onto the end of the triphosphate chain provides for a large class of modified dNTPs, such as those set forth in FIGS. 13 through 20 and throughout the present disclosure.

For example, FIGS. 13A, 13B, 13C and 13D are embodiments of modified dNTPs that are useful for enhancing signals of incorporation events in molecular sensors based on polymerase enzymes. FIG. 13A is an example of chlorine substitution at the 6-position of the purine. FIG. 13B is an example of sulfoxide derivatization of the α-phosphate group of the triphosphate. FIG. 13C is an example of sulfoxide derivatization of the 2-position of the pyrimidine. Lastly, FIG. 13D is an example of bromine substitution at the 5-position of the pyrimidine.

Additional examples of modified dNTPs usable to enhance signals from a biosensor comprising a polymerase enzyme are set forth in FIGS. 14A and 14B. The modified dNTPs illustrated are “deaza” purines, meaning that a nitrogen atom in the purine, the 7-nitrogen, is replaced with a carbon atom (in these examples, at the location of the “star”). FIG. 14A illustrates the chemical structure of 7-deaza-2′-deoxyadenosine-5′-triphosphate. FIG. 14B illustrates the chemical structure of 7-deaza-2′-deoxyguanosine-5′-triphosphate. These molecules are depicted as the tetra-lithium salts, although other salts, including mixed salts, may be usable as modified dNTPs in incorporation events by a polymerase.

FIGS. 15A, 15B and 15C depict further examples of modified dNTPs comprising various derivatizations at the γ-phosphate of the triphosphate of a standard dCTP. These modified compounds each feature groups added to the γ-phosphate, extending each to a hexa-phosphate with various ether linkages (e.g., PEG) as tethers extending off the end phosphate group in the hexa-phosphate chain. The compound of FIG. 15A further comprises a DBCO moiety usable for further derivatization using “Click Chemistry.” DBCO (or ADIBO) is the acronym for the substituent, azadibenzocyclooctyne, which can be seen at the far left of the molecule in FIG. 15A. The compound in FIG. 15A is obtained by reaction of the suitable DBCO-PEG linker with the hexa-phosphate modified dCTP. The reagents for Click Chemistry are obtainable from BroadPharm, San Diego, Calif., amongst other suppliers. The alkyn triple bond in the azacyclooctane ring is then available for further derivatization of the compound of FIG. 15A (such as to produce the compounds of FIGS. 15B and 15C).

The modified dCTP depicted in FIG. 15A carries a charge of −6 due to the presence of the hexa-phosphate group between the PEG moiety and the deoxyribose moiety. The “+” sign and arrow over the top of the molecule indicate the portion of the molecule added to the native dCTP molecule.

The modified dCTP depicted in FIG. 15B is derived from the compound in FIG. 15A by reaction of the triple bond on the azacyclooctyn ring with the appropriately substituted azide to form the triazole-azacyclooctane fused ring system as shown.

Further, the modified dCTP depicted in FIG. 15C is derived from the compound in FIG. 15B by reaction with the appropriately substituted amide, displacing the quaternized moiety as a neutral leaving group.

One benefit of using modified dNTPs, (such as those illustrated in FIGS. 13A-13D, FIGS. 14A and 14B, and FIGS. 15A-15C, amongst others), to enhance signals from polymerase-based sensors is that labeling of the template DNA, synthesized DNA, or dNTPs is not necessary, nor is the use of any other detectable labels. The use of the modified dNTPs herein, on the other hand, modify the conductivity properties of the polymerase-bridge complex, and thereby directly enhance the resulting signals produced by the complex during the enzyme activity. This is a benefit relative to other labeling methods in which the means of detecting the label dominate, constrain, complicate or limit such approaches.

Specific experiments using representative modified dNTPs with the molecular electronic sequencing sensor of FIGS. 1-4 were shown in FIGS. 7, 8, and 9 (exemplifying the use of deaza-modified dGTPs), and in FIG. 10 (exemplifying the use of γ-phosphate modified dCTP-Cy7 and dCTP-Lactose).

In various embodiments of the present disclosure, the use of chemically modified dNTPs enhance the electrical signal parameters of a molecular electronic sensor comprising a polymerase enzyme.

In other embodiments, modified dNTPs may only achieve beneficial or greater levels of signal enhancement in a system that also comprises the use of a particular native or mutated or chemically modified polymerase enzyme, or the use of an appropriate buffer, or the use of an appropriate bridge molecule.

In various embodiments, the pH and/or chemical makeup of a buffer in a test solution comprising a modified dNTP and provided to a sensor may influence the charge on various groups on modified dNTPs such as phosphate groups, other ionizable groups e.g., sulfonate, or amine groups that may be protonated. For example, phosphate groups on a modified dNTP may be all negatively charged or may be partially or fully protonated as —OH groups. In accordance to the present disclosure, various phosphate groups in tri-, tetra-, penta-, hexa-, etc. phosphate chains, may be shown as salts, partial salts, or with each of the phosphate groups protonated to —OH groups in the various drawing figures. The understanding is that what is depicted in a drawing figure for the charges on a polyphosphate chain is not limiting, and the various phosphate groups may be all negatively charged, partially protonated or fully protonated (resulting in all —OH groups). Further, the counterion on any negatively charged phosphate group oxygen atom is also not limited, and may be any M⁺ species (e.g., Li⁺), M²⁺ species (e.g., Mg²⁺), or any ammonium salt, (e.g., R₃NH⁺). Similarly, sulfonic acid groups may be shown in their acid form (—SO₃H), or as the deprotonated sulfonate anion (—SO₃⁻).

In various embodiments, an appropriate combination of dNTP modification, polymerase modification, buffer modification and bridge molecule may result in desirable signal enhancement. Similarly, modified dNTPs for signal enhancement can potentially provide for additional enhancement through the use of such modified polymerases, modified buffers, or modified bridges. An additional benefit of the present disclosure is that additional signal enhancement is possible by optimization of these other major system parameters, relative to various embodiments of modified dNTPs.

FIGS. 16, 17 and 18 illustrate general classes of modified dNTPs usable for enhanced signaling in biosensors comprising a polymerase enzyme. The various substituent groups found in these general compounds [16], [17], and [18], respectively, are selected independently, and these substituent selections may be different for a particular nucleotide base (“Nuc”) in order to provide for base discrimination in the electrical signals produced in DNA sequencing applications. In various aspects, the substituents (R¹, R², R³, R⁴, R⁵, L¹, L², and Y, as variously set forth in compounds [16], [17] and [18]) may be independently selected to produce dNTPs capable of providing enhanced signaling according to any one or combination of the following mechanisms:

(1) Through direct interaction of negative charges or positive charges on a substituent with the conducting portion of the molecular sensor. Specific embodiments are shown without limitation in FIGS. 30A-30D;

(2) Through direct π-π and hydrophobic interaction of aromatic rings, provided by one or more of the substituents, with the conducting portion of the molecular sensor. Specific embodiments are shown without limitation in FIGS. 31A-31D;

(3) Through both charge and π-π interactions, e.g., with a N-alkyl-pyridinium (positive) substituent or pyrene sulfonate (negative) substituent. Specific embodiments are shown without limitation in FIGS. 32A and 32B;

(4) Through electrochemical reduction and oxidation of R¹ (e.g., for reversibly oxidizable groups such as ferrocene or hydroquinone). Specific embodiments are shown without limitation in FIGS. 33A and 33B;

(5) When R¹ contains a conducting molecular component such as a graphene nano ribbon, a polythiophene polymer, or poly aromatic hydrocarbon ring ribbon structure, and the sensor contains two disconnected conductors (each linked to a different metal electrode), through creating and breaking a conductive link between said two conductors. Specific embodiments are shown without limitation in FIG. 34;

(6) When R¹ is a long, rigid molecule that is narrow near the phosphate end and wide at the other end, through steric interaction of the wide end with the conductor that stretches out the linker from polymerase to conductor, altering the interaction between the linker and the conductor in a manner dependent on the length of R¹. In various aspects, the linker would contain multiple groups such as aromatic rings that could bind to the conductor and alter conductivity upon association or dissociation. With multiple groups on the linker, it will be possible to observe no dissociation of the groups from the conductor for a short linker, partial dissociation of some groups for an intermediate length linker, and complete dissociation of groups for a sufficiently long linker—each with a distinct electrical signal. The rigidity and wide end of R¹ ensures that the conductor is contacted and pushed away from the polymerase when the molecule binds so that partial or complete dissociation of groups occurs for sufficiently long R¹. Specific embodiments are shown without limitation for R¹ and linkers between conductor and polymerase in FIGS. 35A and 35B;

(7) Indirectly, by altering the conformational change of the polymerase during incorporation of a modified dNTP. In various embodiments, R¹ may bind to a specific site on the polymerase near the dNTP or DNA binding site, or may alter the linker's interaction with the polymerase. Changes in the substituents n and Y can also affect the time dependence or kinetics of the conformational changes in the polymerase, which could amplify signals when the electrical conductivity between electrodes is sensitive to conformational changes. Specific examples of dNTP derivative structures are shown without limitation in FIGS. 36A and 36B.

The various substituent selections will be further understood when specific species of modified dNTPs are presented and discussed.

In reference to the general chemical structure [16] in FIG. 16, Nuc represents a DNA base, such as unmodified A, T, C, or G, and n is an integer >2, such as to produce a number of species compounds comprising tri-, tetra-, penta-, hexa-, and so forth polyphosphate chain lengths. Y may be the native oxygen of a dNTP or a tolerated alternative such as sulfur, boron or iodine. The terminal group R¹ that caps the last phosphate group in the chain may comprise any substituent capable of achieving at least one of the functional categories listed numerically above and discussed herein. The choices for n, Y, and R¹ are made independently, and may be different for each nucleotide base in order to provide for base discrimination in the electrical signals produced in DNA sequencing applications. In compound [16], R¹ is selected from H, alkyl (e.g., linear or branched C₁-C₅), cycloalkyl (e.g., C₃-C₈), —(CH₂CH₂O)ₓMe wherein x is an integer 1 to about 20, or aryl, wherein any alkyl is branched to any degree and any alkyl, cycloalkyl or aryl substituent is optionally substituted with halogen, Me, or OMe; and Y=OH, SH, or BH₃, with the proviso that if Y=O and n=2, then R¹ is not H, (in other words, the scope of compound [16] excludes the natural dNTPs).

Classes of Modified dNTPs

FIG. 17 illustrates another general class of modified dNTPs useful for signal enhancement in molecular sensors comprising a polymerase enzyme. These compounds [17] allow for Y as a modification to the DNA backbone, a polyphosphate chain with n>1, and a general group R¹ with linking/binding bivalent groups L¹ and L², and with an additional allowed modification via the substituent R³. In compound [17], R¹ is selected from H, alkyl (e.g., linear or branched C₁-C₅), cycloalkyl (e.g., C₃-C₈), —(CH₂CH₂O)ₓMe wherein x is an integer 1 to about 20, or aryl, wherein any alkyl is branched to any degree and any alkyl, cycloalkyl or aryl substituent is optionally substituted with halogen, Me, or OMe. Also in compound [17], Y=OH, SH, or BH₃, and Nuc is a DNA base, such as unmodified A, T, C, or G. Further for compound [17], bivalent moeities -L¹- and -L²- are, optionally and independently, a covalent bond, a linker/spacer such as —(CH₂)_{q}— (wherein q is an integer from 1 to about 10), —CH₂CH₂(OCH₂CH₂)_{y}— (wherein y is an integer from 1 to about 8), —(CH₂)_{q}—O—(CH₂CH₂O)_{y}—CH₂— (wherein q is an integer from 1 to about 10 and y is an integer from 1 to about 8), —CO(CH₂)ᵣ— (wherein r is an integer from 1 to about 10), —COCH₂CH₂(OCH₂CH₂)_{z}— (wherein z is an integer from 1 to about 6), —COCH₂CH₂CONH(CH₂)ₘ— (wherein m is an integer from 1 to about 6), —COCH₂CH₂CONH(CH₂CH₂O)ₚCH₂CH₂— (wherein p is an integer from 1 to about 6), 1,4-benzenediyl, 1,3-benzenediyl, or 1,2-benzenediyl, with carbon atoms optionally and independently substituted with halogen, Me, Et, OH, OMe, or CF₃, and with any carbon atom(s) optionally and independently replaced with nitrogen atom(s).

With continued reference to the chemical compound [17] in FIG. 17, L¹ and/or L² may contain a group that can be formed through “click chemistry”, such as a 1,2,3-triazole ring (e.g., formed from alkyne+azide). The 1,2,3 triazole can optionally be fused to one or more additional rings, preferably including an 8-membered ring/triazole fusion. In compound [17], the endcap group R² is selected from alkyl (e.g., linear or branched C₁-C₂₀), cycloalkyl (e.g., C₃-C₁₂), aryl (including polycyclic substituents up to 10 rings), heteroaryl (including polycyclic substituents up to 10 rings), ferrocene, oligothiophene, heteroaryl-alkyl, aryl alkyl, optionally and independently substituted with halogen, alkyl (linear or branched C₁-C₁₀), O-alkyl (linear or branched C₁-C₁₀), CF₃, CHF₂O, RSO₂, amine, or amide. R² also includes oligosaccharides such as various cyclodextrins, optionally substituted with O-alkyl (linear or branched C₁-C₁₀), O-benzyl, O-sulfate, methyl-(PEG)ₙ (wherein n is from about 1 to about 20) or —O₂C-alkyl (linear or branched C₁-C₈). R³ is selected from H or a halogen such as F.

FIG. 18 depicts the general compound [18] representing another class of modified dNTPs usable to enhance signaling in polymerase-based biosensors. In these compounds [18], ketone (or alternatively, aldehyde) and alkoxyamine chemistry are used to form the various molecules. The genus compound [18] allows for Y substitution as a modification to the DNA backbone, a chain of n>1 phosphates, and a general group R¹ with linking/binding groups L², and with additional allowed modification R³, and a pair of groups R⁵ and R⁴. In particular, these forms may be obtained when ketone (or aldehyde) and alkoxyamine chemistry are used to form the molecule.

In reference now to the general structure [18], R¹ is selected from H, alkyl (e.g., linear or branched C₁-C₅), cycloalkyl (e.g., C₃-C₈), —(CH₂CH₂O)ₓMe wherein x is an integer 1 to about 20, or aryl, wherein any alkyl is branched to any degree and any alkyl, cycloalkyl or aryl substituent is optionally substituted with halogen, Me, or OMe. R³ is selected from H or halogen. Further in compound [18], Y=OH, SH, or BH₃ and Nuc is a DNA base, such as unmodified A, T, C, or G. R⁴ and R⁵ in compound [18] are independently selected from H, alkyl (e.g., linear or branched C₁-C₂₀), cycloalkyl (e.g., C₃-C₁₂), aryl (including polycyclic substituents up to 10 rings), heteroaryl (including polycyclics up to 10 rings), ferrocene, oligothiophene, heteroaryl-alkyl, aryl alkyl, optionally and independently substituted with halogen, alkyl (linear or branched C₁-C₁₀), O-alkyl (linear or branched C₁-C₁₀), CF₃, CHF₂O, RSO₂, amine, or amide. R⁴ and R⁵ also include, independently, oligosaccharides including various cyclodextrins, optionally substituted with O-alkyl (linear or branched C₁-C₁₀), O-benzyl, O-sulfate, methyl-(PEG)ₙ (wherein n is from about 1 to about 20) or —O₂C-alkyl (linear or branched C₁-C₈).

With continued reference to FIG. 18 and genus compound [18], L² is selected as per L¹ and/or L² in compound [17]. In various embodiments, -L²- is selected from a covalent bond, a linker/spacer such as —(CH₂)_{q}— (wherein q is an integer from 1 to about 10), —CH₂CH₂(OCH₂CH₂)_{y}— (wherein y is an integer from 1 to about 8), —(CH₂)_{q}—O—(CH₂CH₂O)_{y}—CH₂— (wherein q is an integer from 1 to about 10 and y is an integer from 1 to about 8), —CO(CH₂)ᵣ— (wherein r is an integer from 1 to about 10), —COCH₂CH₂(OCH₂CH₂)_{z}— (wherein z is an integer from 1 to about 6), —COCH₂CH₂CONH(CH₂)ₘ— (wherein m is an integer from 1 to about 6), —COCH₂CH₂CONH(CH₂CH₂O)ₚCH₂CH₂— (wherein p is an integer from 1 to about 6), 1,4-benzenediyl, 1,3-benzenediyl, or 1,2-benzenediyl, with carbon atoms optionally and independently substituted with halogen, Me, Et, OH, OMe, or CF₃, and with any carbon atom(s) optionally and independently replaced with nitrogen atom(s). Further, L² may contain a group that can be formed through “click chemistry”, such as a 1,2,3-triazole ring (e.g., formed from alkyne+azide), and may comprise the options depicted in FIG. 19 for L¹.

FIG. 19 illustrates non-limiting embodiments for the bivalent linker substituent, L¹, usable in the genus compound [17] in FIG. 17. These options also double as possibilities for L² in compound [18] of FIG. 18. As shown, the various examples include two different triazole/dibenzoazacyclooctane fused ring systems [19a] and [19b], and a triazole/cyclooctane/cyclopropane fused ring system [19c]. As discussed, the triazole in these and other structures may be formed by the reaction between an azide and the alkyn moiety in an cyclooctyne ring, such as exemplified in click chemistry.

Various embodiments of modified dNTPs based on dCTP are set forth in FIGS. 20A-20F. The embodiments shown in FIGS. 20B-20F further comprise a 1,2,3-triazole moiety. These exemplary modified dCTPs are shown for the purpose of illustration only, and are not meant to limit the scope of the modified dNTPs disclosed herein. For example, dNTPs such as these may be based on other dNTPs besides dCTP, and/or may comprise different appendages, such as other chain lengths for any of the repeating units (—(CH₂)ₓ—, PEG, polyphosphate, etc.). The compounds in FIGS. 20A-20F comprise specific C-tetra-phosphates, with diverse groups added to the terminal phosphate via a DBCO click-chemistry linker. FIG. 20B and FIG. 20F further exemplify γ-phosphate modification in the compounds dCTP-Cy7 and dCTP-Lactose, respectively, used in sensor experiments that generated the current versus time plot of FIG. 10. The various groups added in these modified dNTPs provide for different charges (DBCO, Cy7, pipDMA), different sizes (e.g., molecular length) (PEG9), and different polar forms (TPMD, Lactose).

A polymerase extension functional assay shows that polymerase can incorporate these and other modified dNTPs. FIG. 21 shows a gel image from such a polymerase extension assay. To arrive at this result, primed, single-stranded template is incubated with polymerase and different dNTP mixtures. If the enzyme can incorporate and extend the dNTPs, double-stranded DNA product is produced, and results in a band in the corresponding gel lane. The experimental conditions for this primer extension assay were as follows:

Template: 1 μM of single stranded template DNA, with primer annealed;

Template sequence: (70 bases, poly ACTG): 5′-CGC CGC GGA GCC AAG ACTG ACTG ACTG ACTG ACTG ACTG ACTG ACTG ACTG ACTG TTG CAT GTC CTG TGA-3′ (SEQ ID NO: 6);

Primer sequence: (15 bases): 5′-TCA CAG GAC ATG CAA-3′ (SEQ ID NO: 7)

Buffer: 10 mM Tris, 10 mM MgCl₂, 50 mM NaCl;

dNTP concentrations: 2.5 mM of each nucleotide, 10 μM total dNTP concentration;

Enzyme: 5 Units of Klenow exo-;

Conditions: incubation for 30 minutes at 37° C.;

Imaging: 3.5% Agarose gel in TAE with ethidium bromide stain.

Lanes are as delineated in the numerical key below. Lane 4 shows the product from natural dNTPs. Lane 6 uses 4 modified dNTPs. Lane 7, 8, and 9 show three of the γ-phosphate modifications. Thus, all modified dNTPs tested produce DNA product. Lane 5 uses terminator nucleotides (ddNTPs) that cannot be extended, and the result is no product/no band as a negative control. Lanes 2 and 3 are also negative controls, without all reactants required for extension. Lanes 10 and 11 are further negative controls.

The key to the lanes in the polymerase activity assay is as follows:

1—100 base DNA size ladder

2—DAN ONLY (no polymerase or dNTPs)

3—DNA+Klenow polymerase ONLY (no dNTPs)

4—4 dNTPs (native forms)

5—4 ddNTPs (dideoxy terminators)

6—4 modified dNTPs
5-Bromo-2′-deoxycytidine-5′-Triphosphate
7-Deaza-2′-deoxyguanosine-5′-Triphosphate
7-Deaza-2′-deoxyadenosine-5′-Triphosphate
2-Thiothymidine-5′-Triphosphate (2 thio dTTP)

7—dC4P-DMA, other dNTPs native

8—dC4P-DBCP, other dNTPs native

9—dC4P-Cy7, other dNTPSs native

10—dC4P-DMA+Klenow polymerase only, no template DNA

11—dC4P-DMA ONLY, no polymerase

12—Low molecular weight DNA size ladder

The polymerase activity assay in FIG. 21 shows that three of these forms are functional with polymerase enzyme. The assay shown in FIG. 21 also demonstrates that 4 of the modified dNTPs from FIGS. 13A-13D and FIGS. 14A-14B are also functional. Highly similar considerations apply to the sequencing of RNA, if the polymerase in question is a reverse transcriptase polymerase.

The synthesis of various embodiments are outlined in the Synthetic Organic Chemistry section below:

Synthetic Organic Chemistry

Chemical syntheses used to produce the various modified dNTPs herein are disclosed, including syntheses of the modified dNTPs shown in FIGS. 20A-20F. The functionality of three of these molecules is illustrated in the extension assay results shown in FIG. 21, which shows that the polymerase enzyme can incorporate and extend these molecules. It is further noted that although these molecules below are derived through a process of DBCO-mediate click chemistry to add groups to a primary molecular product, other click chemistries could similarly be utilized as the basis for such families of molecules.

Synthesis of DBCO-PEG-OH (Compound [IV])

See FIG. 22 for a synthesis of DBCO-PEG-OH (compound [IV]).

To a solution of 2-(2-(2-aminoethoxy)ethoxy)ethan-1-ol (compound [I], 0.267 g, 1.789 mmol) in 2 ml anhydrous DCM at RT was added slowly a solution of DBCO-NHS (compound [II], 0.24 g, 0.596 mmol) in 2 ml anhydrous DCM. After completion of addition, the reaction solution was stirred at RT for 2 hours. HPLC indicated complete disappearance of the succinimide. The reaction was kept in the dark at −20° C. in a freezer. The next morning, the reaction solution was warmed up to RT and diluted with 3 ml DCM. Silica gel (5 g) was added, and the slurry evaporated to dryness in a rotary evaporator. The residual powder was loaded onto a ISCO loading cartridge and purified by column chromatography (12 g silica gel column, 5-20% methanol/DCM) to afford 0.2 g of DBCO-PEG-OH (compound [IV]). Yield: 77%

¹H NMR (499 MHz, Chloroform-d) δ 7.65 (dd, J=7.6, 1.3 Hz, 1H), 7.53-7.42 (m, 1H), 7.42-7.17 (m, 6H), 6.41 (t, J=5.7 Hz, 1H), 5.12 (d, J=13.9 Hz, 1H), 3.75-3.49 (m, 10H), 3.44 (dddd, J=28.0, 10.0, 6.3, 4.0 Hz, 2H), 3.36-3.21 (m, 2H), 2.78 (ddd, J=16.8, 8.5, 6.2 Hz, 1H), 2.41 (ddd, J=14.8, 8.5, 6.1 Hz, 1H), 2.16 (dt, J=15.1, 6.2 Hz, 1H), 2.01-1.86 (m, 1H).

Mass: calculated for C₂₅H₂₈N₂O₅, [M]: 436.20, observed: [M+23] 459.5 in positive mass.

HPLC: 10 minutes HT-LC-MS method, retention time for product: 6.5 minutes.

Synthesis of DBCO-PEG-Monophosphate (Compound [V])

See FIG. 23 for a synthesis of DBCO-PEG-monophosphate (compound [V]).

DBCO-PEG-OH (compound [IV], 35.8 mg, 0.082 mmol) was co-evaporated with anhydrous acetonitrile (2×1 ml) and then was dissolved in trimethylphosphate (0.42 ml). Phosphorous oxychloride (POCl₃, 16 μL, 0.64 mmol) was added to this cooled and stirred solution, and the reaction mixture was stirred for 2 hours. This reaction mixture was added dropwise over 5 minutes to tributylammonium pyrophosphate (1 equiv, 0.082 mmol, 0.5M solution in anhydrous DMF) and tributylamine (76 mg, 0.41 mmol) was added and stirred for 60 minutes. 5 ml TEAB (0.1M) buffer was added to quench the reaction. Solvent was removed under vacuum and the resulting residue was kept in a refrigerator overnight. The next morning, the residue was purified by C18 ISCO column (15.5 g C18 column, 0-100% acetonitrile/0.1M TEAA in water) to yield approximately 45 mg compound [V]. Residual trimethylphosphate prevented any meaningful NMR spectrum. The compound [V] was directly used in the next step for the synthesis of compound [VIII].

HPLC: 10 minutes HT-LC-MS method, retention time for starting material DBCO-PEG-alcohol: 6.5 minute: retention time for product: a group of three peaks: 5.0 minutes, 5.2 minutes and 5.5 minutes.

Mass: calculated for C25H31N2O14P3, [M]: 676.4, observed: [M−1] 675.3 in negative mass.

Synthesis of dC-P4-Click (Compound VIII)

See FIGS. 24A-24B for a synthesis of dC-P4-click (compound [VIII]).

DBCO-PEG-monophosphate (compound [V], 45 mg, 0.06 mmol) was co-evaporated with anhydrous acetonitrile (2×1 ml) and then dissolved in anhydrous DMF (1.0 ml). Carbonyldiimidazole (compound [VI], 4 equiv., 38.7 mg, 0.24 mmol) was added and the reaction mixture was stirred at room temperature for 4 hours. Methanol (6 equiv., 14.7 μL) was then added and stirring was continued for 30 minutes. To the reaction mixture a solution of dCTP (bis)tributylammonium salt (70.2 mg, 0.084 mmol) in 0.5 ml DMF and MgCl₂ (57 mg, 0.6 mmol) were added. The resulting mixture was stirred overnight. The next morning, HPLC indicated the shift from the less polar starting material DBCO-PEG-triphosphate to more polar reaction products. The crude product was purified by reverse phase C18 column on ISCO (15.5 g C18 column, 0-100% ACN/0.1M TEAA in HPLC grade water. There were a group of peaks eluting at around 30-40% ACN/0.1M TEAA in HPLC grade water. The first two fractions P1(f26+f27) were collected, solvent was removed, and the residue was dried under vacuum to give 9.2 mg compound [VIII]. The middle two fractions P2 (f28+f29) were collected, solvent was removed, the residue was dried under vacuum to give an additional 10.7 mg compound [VIII]. The last two fractions P3 (f30+f31) were collected, solvent was removed, and the residue was dried under high vacuum to give a further 6.5 mg compound [VIII]. By mass, the first peak is mainly d-C-P4-click, the second is a mixture of d-C-P4-click and traces of d-C-P5-click, d-C-P6-click and d-C-P7-click.

HPLC: 10 minutes HT-LC-MS method, retention time for starting material: a group of three peaks: 5.0 minutes, 5.2 minutes and 5.5 minutes. Retention time for product CP4-click: 4.8 and 4.9 minutes, two peaks.

¹H NMR (500 MHz, Deuterium Oxide) δ 7.96 (t, J=8.5 Hz, 1H), 7.66 (d, J=7.4 Hz, 1H), 7.60-7.30 (m, 7H), 6.33 (t, J=6.7 Hz, 1H), 6.15 (q, J=7.8 Hz, 1H), 5.09 (d, J=14.4 Hz, 1H), 4.78 (s, 100H), 4.67-4.53 (m, 1H), 4.21 (d, J=5.0 Hz, 4H), 4.13 (d, J=7.1 Hz, 2H), 3.83 (d, J=14.4 Hz, 1H), 3.75 (q, J=7.1, 6.0 Hz, 2H), 3.69 (q, J=6.5, 5.2 Hz, 2H), 3.63-3.54 (m, 2H), 3.47 (dt, J=10.6, 5.3 Hz, 1H), 3.38 (dq, J=11.3, 5.9 Hz, 1H), 3.24-3.05 (m, 54H), 2.53 (dt, J=15.5, 5.8 Hz, 1H), 2.44-2.32 (m, 1H), 2.24 (tdq, J=20.8, 14.3, 7.0 Hz, 4H), 1.91 (d, J=1.2 Hz, 9H), 1.26 (td, J=7.4, 1.0 Hz, 82H).

Phosphorus NMR: −11.0 (m, integration 100), −22.4 (m, integration 100).

Mass Spec. M=965 negative ion: calculated for M-H: 964.6 Observed: 964.3.

Synthesis of dC-P4-Pip-DMA (Compound [X])

See FIGS. 25A-25B for a synthesis of dC-P4-pip-DMA (compound [X]).

MIR96-IN-1-azide (compound [IX], 3.7 mg, 6.28 μmop and dC-P4-click (compound [VIII], 7.5 mg, 5.9 μmop were mixed in 0.3 ml of 1:1 water/acetonitrile and the reaction mixture was stirred at RT overnight. HPLC indicated disappearance of the starting material dCP4-click and the formation of some new less polar products. Crude mass indicated the formation of desired compound [X]. Solvent was removed and the residue was dried under vacuum to afford 8.6 mg crude compound [X].

HPLC: 10 minutes HT-LC-MS method, retention time for starting material dCP4-click: 4.8 and 4.9 minutes, two peaks, retention time for MIR96-azide: 9.6 minute, retention time for click adduct: 5.49 minute.

¹H NMR (500 MHz, Methanol-d₄) δ 8.12 (d, J=7.3 Hz, 1H), 8.01 (dot, J=8.7, 3.2 Hz, 3H), 7.60 (d, J=7.5 Hz, 1H), 7.51 (dd, J=20.4, 11.8 Hz, 4H), 7.43-7.20 (m, 2H), 7.16 (d, J=8.1 Hz, 1H), 7.02 (d, J=9.3 Hz, 1H), 6.38-6.19 (m, 1H), 6.02 (t, J=16.7 Hz, 0H), 5.85 (d, J=16.2 Hz, 0H), 4.67-4.51 (m, 1H), 4.51-4.21 (m, 3H), 4.07 (s, 2H), 3.78 (t, J=7.4 Hz, 1H), 3.73-3.32 (m, 9H), 3.27-3.07 (m, 25H), 3.09-2.70 (m, 6H), 2.66 (s, 1H), 2.33 (q, J=9.4, 8.5 Hz, 1H), 2.27-1.95 (m, 3H), 1.82-1.53 (m, 2H), 1.54-1.35 (m, 18H).

Phosphorus NMR: −10.63 (m, integration 100), −22.06 (m, integration 108).

Mass: M=1554.4 negative ion: calculated for M−1: 1553.4 is M−1, observed: 1552.8 calculated for M+Na−2H: 1575.4 Observed: 1574.8.

Synthesis of dCP4-Cy7 (Compound [XII])

See FIGS. 26A-26B for a synthesis of dCP4-Cy7 (compound [XII]).

Cy7-azide (compound [XI], 5.0 mg, 4.37 μmol) and dCP4-click (compound [VIII], 7.5 mg, 5.9 μmol) were mixed in 0.3 ml of 1:1 water/acetonitrile solution and stirred at RT overnight. HPLC indicated the disappearance of the starting material dCP4-click and the formation of some new products. There was still some excess Cy7-azide [XI] present in the reaction mixture. Crude mass indicated formation of the desired product. Solvent was removed and the residue was dried under vacuum to afford 7.3 mg of compound [XII].

HPLC: 10 minutes HT-LC-MS method, retention time for starting material dCP4-click: 4.8 and 4.9 minutes, two peaks, retention time for Cy7-azide: 4.59 minutes, retention time for click adduct: 4.48 minute.

¹H NMR (500 MHz, Methanol-d₄) δ 7.96-7.76 (m, 1H), 7.74 (d, J=1.7 Hz, 1H), 7.67-7.42 (m, 1H), 7.41-7.21 (m, 2H), 7.17-7.01 (m, 1H), 6.42 (d, J=14.0 Hz, 1H), 4.53 (d, J=37.3 Hz, 1H), 4.38-3.99 (m, 3H), 3.80-3.40 (m, 3H), 3.28-3.11 (m, 17H), 3.08-2.97 (m, 1H), 2.94 (t, J=6.8 Hz, 2H), 2.78 (t, J=6.4 Hz, 2H), 2.60-2.43 (m, 1H), 2.21 (p, J=7.1 Hz, 3H), 2.11-1.97 (m, 1H), 1.74 (p, J=6.8 Hz, 1H), 1.22 (t, J=4.3 Hz, 6H).

Phosphorus NMR: too little material was available to obtain a reasonable phosphorus NMR. The signals were very weak. However, the desired product was evident from the mass spectral data.

Mass: M=2021.9 Negative ion Calculated for M-6H+5Na: 2130.9 Observed: 2134 for neg. Positive ion Calculated for M-3H+5Na: 2132.9. Observed: 2136 for positive. (Note: 13C and 2H increase observed masses).

All of the HPLCs were taken with 10 minute: HT-LCMS method with ammonium acetate as the additive. Solvent: acetonitrile and water with 25 mM ammonium acetate. Method: 0-0.5 minute: 5% acetonitrile/water, 0.5-6.5 minute: 5-95% acetonitrile/water, 6.5-9 minute: 95% acetonitrile/water, 9-9.5 minute: 95%-5% acetonitrile/water, 9.5-10 minute: 5% acetonitrile/water.

Synthesis of dCP4-TPMD (Compound [XIV])

See FIGS. 27A-27B for a synthesis of dCP4-TPMD (compound [XIV]).

TMPD-azide-2HCl (compound [XIII], 2.28 mg, 8.85 μmol) and dCP4-click (compound [VIII], 7.5 mg, 5.9 μmol) were mixed in 0.38 ml of 1:1 water/acetonitrile and the reaction mixture was stirred at RT overnight. Acetonitrile was removed and the concentrated material loaded onto a 4 g C18 column directly and purified using 0-100% ACN/0.1M TEAA in water. There was a large peak was observed to elute around 32% ACN/0.1M TEAA in water. Solvent was removed from the eluent and the residue dried further under vacuum to afford 4.9 mg compound [XIV].

Synthesis of dCP4-Lactose (Compound [XVI])

See FIGS. 28A-28B for a synthesis of dCP4-Lactose (compound [XVI].

2-Azidoethyl-β-D-lactopyranoside (compound [XV], 3.23 mg, 10.4 μmol) and dCP4-click (compound [VIII], 10 mg, 7.86 μmol) were mixed in 0.5 ml of 1:1 water/acetonitrile and the reaction mixture was stirred at RT overnight. Acetonitrile was removed and the concentrated material loaded to a 4 g C18 column directly and purified using 0-100% ACN/0.1M TEAA in water. There was a large peak observed to elute around 25% ACN/0.1M TEAA in water. Solvent was removed from the eluent and the residue dried under vacuum to afford 10.4 mg of compound [XVI]. Mass from HT lab confirms desired mass was observed. HPLC indicates the product has a different retention time from the starting material dCP4-Click [VIII].

Synthesis of dCP4-PEG9 (Compound [XVIII])

See FIGS. 29A=29B for a synthesis of dCP4-PEG9 (compound [XVIII]).

PEG9-azide (compound [XVII], 3.4 mg, 8 μmol) and dCP4-click (compound [VIII], 7.5 mg, 5.9 μmol) were mixed in 0.38 ml of 1:1 water/acetonitrile and the reaction mixture was stirred at RT overnight. Acetonitrile was removed and the concentrated mixture loaded to a 4 g C18 column directly and purified using 0-100% ACN/0.1M TEAA in water. A large peak was observed to elute with 33% ACN/0.1M TEAA in water. After removing solvent, and drying under vacuum for 2 hours, 5.0 mg compound [XVIII] was obtained. Mass spec confirmed the identity as compound [XVIII]. HPLC showed a different retention time from the starting material dCP4-Click [VIII].

### Further Embodiments and Considerations of Modified dNTPs

With reference now to FIG. 30A and FIG. 30B, examples of R¹ that provide a positive charge at the end of a dNTP are depicted. Either of these exemplary R¹ moieties may be used in the generic structures [17], [18] or [19] (see FIGS. 16-19). These R¹ options provide for unique electrical signal generation for an incorporation event through direct interaction of positive charges on R¹ with the conducting portion, (e.g., the bridge molecule), of a molecular sensor. In FIG. 30A, the positive charge(s) are pH dependent, and are located at either or both of the tertiary nitrogen atoms by protonation, as shown in the partial structure below:

[chemical structure]

Similarly in FIG. 30B, the positive charge on the R¹ group of the dNTP is pH dependent, and is located at the tertiary nitrogen by protonation, as shown in the partial structure below:

[chemical structure]

With reference now to FIG. 30C and FIG. 30D, examples of R¹ that provide a negative charge to the signaling end of a dNTP are depicted. Either of these exemplary R¹ moieties may be substituted into the generic structures [17], [18] or [19] (see FIGS. 16-19). These R¹ options provide for unique electrical signal generation through direct interaction of negative charges on R¹ with the conducting portion, (e.g., the bridge molecule), of a molecular sensor. The R¹ moiety in FIG. 30C provides a single negative charge when the sulfonic acid group is deprotonated to the sulfonate anion (i.e., SO₃−). Similarly, the R¹ moiety in FIG. 30D is capable of providing 4 negative charges when all four of the sulfonic acid groups are each deprotonated to their respective sulfonate anions, recognizing that the quaternized nitrogen in the indole carries a permanent positive charge, and that the overall molecule carries a maximum of a −3 charge when carried in a suitable buffer. In various buffer conditions, the modified dNTP carrying the R¹ substituent of FIG. 30D may have an overall charge of 0, −1, −2 or −3.

FIGS. 31A-31D illustrate four separate options for R¹ that provide a mechanism for unique electrical signal generation through direct π-π and hydrophobic interactions of aromatic rings on R¹ with the conducting portion (e.g., the bridge molecule) of a molecular sensor. In general, these substituents comprise an aromatic ring system (benzene, naphthalene, anthracene, etc.) in combination with a tether comprising mostly PEG units. The length of the tether may be adjusted by addition or subtraction of units, such as to optimize the likelihood that the aromatic signaling group will interact with the conducting portion of the sensor when the modified dNTP is incorporated by the polymerase.

FIGS. 32A-32B illustrate two further options for R¹ that provide a mechanism for unique electrical signal generation through both charge and π-interaction. The example in FIG. 32A includes an N-alkylpyridinium moiety (positive charge), while the example in FIG. 32B includes a pyrene sulfonate moiety (negative charge).

FIG. 33A illustrates an option for R¹ that provides a mechanism for unique electrical signal generation through electrochemical oxidation. In this example, the 1,4-benzoquinone moiety on R¹ is reducible to the corresponding radical anion, which can interact with the conducting portion of the sensor when such a modified dNTP is incorporated by the polymerase. Similarly, FIG. 33B illustrates an option for R¹ that provides a mechanism for unique electrical signal generation through electrochemical oxidation. In this example, the ferrocene (Fe(n⁵-C₅H₅)₂) moiety present on R¹ undergoes a one-electron oxidation-reduction process to the ferrocenium radical ([Cp₂Fe].⁺, which can interact with the conducting portion of the sensor when such a modified dNTP is incorporated by the polymerase.

FIG. 34 illustrates an option for the R¹ group of compound [16] in FIG. 16. Another way to view this substituent is as a combination of R⁴, R⁵, L² and R¹ of compound [18] in FIG. 18. This substituent, went incorporated into a modified dNTP, provides a mechanism for unique electrical signal generation by creating and breaking a conductive link between two electrodes. The substituent in FIG. 34 comprises a long conducting polythiophene polymer portion (the length of which depends on the integer n=1 to 100). It is presumed that in the absence of any modified dNTP, the polymerase and bridge molecule combined structure allows very little current to flow between source and drain electrodes. It is also presumed that there are left and right conductive paths to the electrodes, such that when spanned by a suitably conductive polymer would provide a distinguishably much higher current. The intent is for the incoming modified dNTP to transiently provide this conducting polymer connection via the R¹ group illustrated. In other words, an otherwise open circuit (comprising polymerase, bridge molecule and electrodes) may be closed when the conducting polymer portion of the R¹ depicted in FIG. 34 lays across the non-conducting regions of the bridge during the incorporation event. Thus, FIG. 34 depicts one suitable family of conducting polymers that may be used to close otherwise open circuits during incorporation events. In various embodiments of R¹, n can be an integer from 1 to about 100, such as to provide a length of R¹ in the range of from about 0.5 nm to about 50 nm. In other embodiments, n may be chosen such that the length of R¹ is from about 1 nm to about 20 nm.

In various embodiments, the bridge molecule may purposely comprise an insulating link, such as cyclohexane-1,4-diyl at the branch point that links the bridge molecule to the polymerase. R¹ of FIG. 34 can create a conductive link across the insulating link during dNTP binding to the active site, after incorporation, or at both times, depending on the integer n and the structure of the linker portion of R¹ between the conductive polymer portion and the polyphosphate of the dNTP (here, shown to be a short PEG-4 span, an oxime functionality, and a tetramethylene portion that would connect to the γ-phosphate O atom. R¹ can similarly create a transient conducting link spanning any other insulating segments of the bridge, completing a conductive path.

FIG. 35A depicts another series of uniquely modified dATP molecules. For these modified dATPs, n is an integer from 1 to about 100. In various examples, n may be chosen such that the length of the repeating portion of the molecule measures from about 0.5 nm to about 50 nm, or from about 1 nm to about 20 nm. The modified dATPs of FIG. 35A provide mechanisms for unique electrical signal generation. The R¹ portion of the dATP molecule, which begins at the last phosphate group in the tetraphosphate moiety and extends to the end of the molecule, comprises a long, rigid “stem” structure that begins sterically narrow near the phosphate linkage and ends sterically large at the opposite end, which is seen to comprise a bulky fused ring system, a revolving amide linkage, and two pyridine substituents. In sensors comprising DNA bridges, graphene nanoribbon bridges, or other bridges that comprise aromatic rings, the wide end of the modified dATP of FIG. 35A sterically interacts with the bridge molecule of the sensor causing the linker portion (i.e., the repeating pyrazine/piperazine subunit) to stretch from polymerase to conducting bridge, altering the interaction between the linker portion and the conducting bridge in a manner dependent on the length of R¹.

FIG. 35B depicts a special tether that can be bonded between the polymerase and the bridge molecule in a molecular sensor for interaction with various modified dNTPs such as the dATPs shown in FIG. 35A. The example in FIG. 35B comprises multiple aromatic rings (benzene, naphthalene, pyrene, etc., disposed as intermittent appendages off the backbone of the tether) that can bind to the conducting bridge and alter conductivity upon association and dissociation. With multiple groups on the tether as shown in FIG. 35B, it is possible to observe no dissociation of the groups from the conducting bridge for a short tether, partial dissociation of some groups for an intermediate length tether, and complete dissociation of groups for a sufficiently long tether—each producing a distinct and distinguishable electrical signal through this interaction with the conducting bridge. Thus such different forms of the tether provide a means for the different A/C/G/T modified dNTPs to produce distinguishable signals. The linker rigidity and distal wide end of the R¹ group ensures that the conducting bridge is contacted and pushed away from the polymerase when this binds so that partial or complete dissociation of groups occurs for sufficiently long R¹.

The combination of the modified dATPs of FIG. 35A and the covalently bonded tether of FIG. 35B provide a unique opportunity for enhanced signally during incorporation of the modified dATP. The sulfide end of the tether of FIG. 35B may be covalently bonded (via a sulfide or disulfide link) to an amino acid of the polymerase. The other end of the tether may be covalently bonded to a bridge molecule via a number of possibilities. For a polyaromatic hydrocarbon conductor bridge molecule (e.g., graphene), the other end of the tether can be covalently linked to the bridge through a C—C bond, an O—C bond, or a S—C bond, e.g., through an aryl link to the conductor (e.g., formed using arendiazonium salt), or through an azirene link or cyclopropyl link to the conductor (formed using nitrene or carbene). For a DNA bridge molecule, the tether can be linked to the 2′ position of a deoxyribose, to a position on a modified base, or to a phosphate group through a P—S or P—C bond.

When a modified dATP of FIG. 35A non-covalently binds in the polymerase active site during incorporation, the modified dATP sterically interacts with the tether of FIG. 35B. The interaction of a modified dATP (FIG. 35A) with the special tether (FIG. 35B) influences signaling current by intermittent interaction with the bridge, or by changing the distance or orientation of the polymerase to the bridge, (e.g., shortening or lengthening the tether via binding to it). In general, a modified dNTP, such as the modified dATPs in FIG. 35A, can be designed to interact with the tether, and thereby alter the conformation or distance of the polymerase relative to the bridge, and by that means modulate the bridge current and produce a signal.

FIG. 36 depicts a conducting bridge/polymerase-tether example, wherein the polymerase-tether provides for an interaction with the modified dNTP of FIG. 35A in a way that alters the conformation of the polymerase and therefore impacts the signal. This modified dNTP thereby provides a mechanism for unique electrical signal generation, indirectly by altering the conformational change of the polymerase during the incorporation event. In general, a modified dNTP such as in FIG. 35 can be designed to interact with the tether, and thereby alter the conformation or distance of the polymerase relative to the bridge, and by that means modulate the bridge current and produce a signal.

FIG. 37 shows several modified dNTPs and the results of a polymerase activity assay for these modified dNTPs. The purpose of this experiment is to show the Klenow polymerase can be active with these diverse chemical modifications.

Additional modified dNTPs include the following compounds comprising thio or borano modifications:

[chemical structure]

As shown by the structures above, the thio and borano modification may be at the α-, β-, or γ-phosphate of the triphosphate of the dNTP. The placement of these small modifications close to the base may enhance signaling of an incorporation even involving the modified dNTP through alteration of enzyme action, or proximity of the modification to the molecular bridge, while still being very well tolerated by the enzyme. Other atomic level modifications incorporating similar numbers of bonds could also be considered beyond sulfur and boron substitution, (e.g., iodine atoms).

Affinity Groups in Modified dNTPs

Further embodiments of the present disclosure comprise the use of an affinity group in a modified dNTP with a corresponding affinity complement positioned on the sensor so as to further enhance the influence of the signaling group on the current through the molecular sensor. In various embodiments, an affinity group is provided between the polyphosphate chain and the signaling group, closer to the signaling group end of the tether. The purpose of this is arrangement is to promote the most impactful positioning of the signaling group relative to the molecular complex of the sensor, and to increase the duration of the interaction of the dNTP with the conducting bridge molecule of the sensor. An affinity complement present on the complex promotes the positioning and residence of a charge group on the modified dNTP to have larger impact on current through the bridge. In certain examples, different affinity groups may be employed on the dNTP for each base of the dNTP (C, G, A, T). The affinity group can be highly specific, such as a single stranded DNA 5-mer that would have affinity to its complementary portion of a DNA oligo used as the molecular bridge of the sensor, or it can just represent a charge affinity, such as a negative charge on the dNTP being attracted to a positive charge on the bridge molecule.

Complementary oligos of including DNA analogs may also be beneficial for this purpose, as then can provide tunable binding energies in shorter oligos. For example, such oligos may comprise RNA, PNA (peptide nucleic acid) or LNA (locked nucleic acid) in place of native DNA, or base analogues such as inosine. For the case of graphene nanoribbon bridge, a group comprising a pyrene could have affinity to the bridge via pi-pi stacking interaction of pyrene and graphene. More generally, a pyrene group attached to the bridge and a pyrene group located on the dNTP would have affinity to each other via π-π stacking. Other affinity groups could use material binding peptides, cognate to a material bound to the bridge, or interacting proteins such as two components of a protein complex, or a small molecule or peptide antigen and a cognate antibody of Fab antibody binding domain conjugated to the bridge, or aptamers. Such bindings would preferably be selected and performed under conditions of weaker binding or transient interaction, as it is not desirable to have these interactions persist for more than the timescale of seconds, and preferably only on the scale of 10's to 100's of milliseconds. One or more affinity complements could reside on the sensor complex, the same, or different for the different dNTP affinity groups. In various embodiments, DNA oligos in the 3-mer to 30-mer range suffice as selectable, specific affinity groups for modified dNTPs, as do oligos that use modified forms of DNA, or DNA analogs that hybridize to DNA.

Modified dNTPs, methods of synthesizing modified dNTPs, and the use of modified dNTPs for enhanced signaling of dNTP incorporation events during DNA sequencing in molecular sensors comprising polymerase are provided. In the detailed description herein, references to “various embodiments”, “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to ‘at least one of A, B, and C’ or ‘at least one of A, B, or C’ is used in the claims or specification, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C.

All structural, chemical, and functional equivalents to the elements of the above-described various embodiments that are known to those of ordinary skill in the art are expressly incorporated herein by reference and are intended to be encompassed by the present claims. Moreover, it is not necessary for a molecule, composition, or use to address each and every problem sought to be solved by the present disclosure, for it to be encompassed by the present claims. Furthermore, no chemical, component, or use in the present disclosure is intended to be dedicated to the public regardless of whether the chemical, component, or use is explicitly recited in the claims. No claim element is intended to invoke 35 U.S.C. 112(f) unless the element is expressly recited using the phrase “means for.” As used herein, the terms “comprises”, “comprising”, or any other variation thereof, are intended to cover a non-exclusive inclusion, such that a chemical, chemical composition, process, method, article, or apparatus that comprises a list of elements does not include only those elements but may include other elements not expressly listed or inherent to such chemical, chemical composition, process, method, article, or apparatus.

We claim:

1. A modified nucleotide comprising a structure represented by compound [16], [chemical structure]
wherein:
Nuc is a DNA base selected from A, T, C, and G;
Y is selected from O, S, B or I;
n is an integer from 2 to 5; and
R¹ is selected from: [chemical structure] [chemical structure] [chemical structure] [chemical structure] [chemical structure]
wherein n=1 to 100,
wherein said modified nucleotide is incorporated by DNA polymerase in replication of a DNA template during DNA sequencing.

2. The modified nucleotide of claim 1, wherein Y is O; Nuc is cytosine; n is 3; and R¹ is:

[chemical structure]

3. The modified nucleotide of claim 1, wherein Y is O; Nuc is cytosine; n is 3; and R¹ is:

[chemical structure]

4. The modified nucleotide of claim 1, wherein Y is O; Nuc is cytosine; n is 3; and R¹ is:

[chemical structure]

5. The modified nucleotide of claim 1, wherein Y is O; Nuc is cytosine; n is 3; and R¹ is:

[chemical structure]

6. The modified nucleotide of claim 1, wherein Y is O; Nuc is cytosine; n is 3; and R¹ is:

[chemical structure]

7. The modified nucleotide of claim 1, wherein Y is O; Nuc is cytosine; n is 5; and R¹ is:

[chemical structure]

8. The modified nucleotide of claim 1, wherein Y is O; Nuc is cytosine; n is 5; and R¹ is:

[chemical structure]

9. The modified nucleotide of claim 1, wherein Y is O; Nuc is cytosine; n is 5; and R¹ is:

[chemical structure]

10. The modified nucleotide of claim 1, wherein Y is O; Nuc is cytosine; n is 5; and R¹ is:

[chemical structure]

11. The modified nucleotide of claim 1, wherein Y is O; Nuc is cytosine; n is 5; and R¹ is:

[chemical structure]

12. The modified nucleotide of claim 1, wherein Y is O; Nuc is cytosine; n is 5; and R¹ is:

[chemical structure]

13. The modified nucleotide of claim 1, wherein Y is O; Nuc is adenosine; n is 3; and R¹ is:

[chemical structure]

14. The modified nucleotide of claim 1, wherein Y is O; Nuc is cytosine; n is 3; and R¹ is:

[chemical structure]

15. The modified nucleotide of claim 1, wherein Y is O; Nuc is adenosine; n is 3; and R¹ is: [chemical structure]
wherein n=1 to 100.

16. The modified nucleotide of claim 1, wherein Y is O; Nuc is adenosine; n is 3; and R¹ is:

[chemical structure]

17. A modified nucleotide comprising a structure represented by compound [18], [chemical structure]
wherein:
Nuc is a DNA base selected from A, T, C, and G;
Y is selected from OH, SH, or BH₃;
n is an integer from 2 to 5;
R³ is selected from H or halogen;
R¹ is selected from H, linear or branched C₁-C₅ alkyl, C₃-C₈ cycloalkyl, or aryl, optionally substituted with halogen, Me or OMe, or —(CH₂CH₂O)ₓMe wherein x is an integer 1 to 20;
L² is selected from —(CH₂)_{q}— wherein q is an integer from 1 to 10, —CH₂CH₂(OCH₂CH₂)_{y}— (wherein y is an integer from 1 to about 8), —(CH₂)_{q}—O—(CH₂CH₂O)_{y}—CH₂— (wherein q is an integer from 1 to about 10 and y is an integer from 1 to about 8), —CO(CH₂)ᵣ— wherein r is an integer from 1 to about 10), —COCH₂CH₂(OCH₂CH₂)_{z}— wherein z is an integer from 1 to 6, —COCH₂CH₂CONH(CH₂)ₘ— wherein m is an integer from 1 to 6, —COCH₂CH₂CONH(CH₂CH₂O)ₚCH₂CH₂— wherein p is an integer from 1 to 6, 1,4-benzenediyl, 1,3-benzenediyl, or 1,2-benzenediyl, with carbon atoms optionally and independently substituted with halogen, Me, Et, OH, OMe, or CF₃, or, [chemical structure]
and
R⁴ and R⁵ are independently selected from H, phenyl, [chemical structure]
wherein n=1 to 100.

18. The modified nucleotide of claim 17, wherein: Nuc is A, T, G, or C; Y is OH; n=3 or 5; R¹ is H; R³ is H; L² is —(CH₂)₄—O—(CH₂CH₂O)₈—CH₂—, and R⁴ is phenyl,

[chemical structure]

19. A method of enhancing an electrical signal generated from a biosensor, the method comprising:
(a) providing a biosensor comprising source and drain electrodes and a polymerase bonded to a bridge molecule bridging the electrodes to complete an electrical circuit;
(b) placing a nucleotide template to be sequenced in communication with the polymerase;
(c) placing a modified dNTP in communication with the polymerase; and
(d) transcribing the nucleotide template by the polymerase,
wherein transcribing comprises incorporating the modified dNTP by the polymerase, and
wherein incorporating the modified dNTP results in an enhanced electrical signal compared to incorporating the corresponding non-modified dNTP.

20. The method of claim 19, wherein the modified dNTP comprises any one of the modified nucleotides of claims 1-18.

21. The method of claim 19, wherein the enhanced electrical signal distinguishes between A, G, C, and T in the nucleotide template.

22. The method of claim 19, wherein the enhanced electrical signal is unique for incorporation of each of a modified dATP, a modified dGTP, a modified dTTP, and a modified dCTP.

23. A method of transcribing a nucleotide template, the method comprising:
(a) providing a polymerase capable of transcribing the nucleotide template;
(b) placing the nucleotide template in communication with the polymerase;
(c) placing modified dNTPs in communication with the polymerase; and
(d) transcribing the nucleotide template with the polymerase by incorporating the modified dNTPs,
wherein the modified dNTPs comprise any one of the modified nucleotides of claims 1-18.
